(19) United States
(12) Patent Application Publication
(10) Pub. No.: US 2023/0372662 A1
(43) Pub. Date: Nov. 23, 2023

(54) STRESS TREATMENT BY NON-INVASIVE, PATIENT-SPECIFIC, AUDIO-BASED BIOFEEDBACK PROCEDURES

(71) Applicant: SOUNDS-U LTD, Tel Aviv (IL)

(72) Inventors: Mordehai RATMANSKY, Tel Aviv (IL); ltai ARGAMAN, Kfar Saba (IL); Yoav SCHWEITZER, Tel Aviv (IL)

(21) Appl. No.: 18/028,185

(22) PCT Filed: Sep. 23, 2021
(86) PCT No.: PCT/IL2021/051163

§ 371 Date: Mar. 23, 2023

(30) Foreign Application Priority Data
Sep. 24, 2020 (US) 63082831

(51) Int. Cl. A61M 21/00; A61B 5/00

(52) U.S. Cl. CPC A61M 21/00; A61B 5/4848; A61M 2021/0027

(57) ABSTRACT

A system and method are provided for treating patient stress, including: receiving sounds vocalized by a patient; determining, from the vocalized sounds, an exceptional frequency that is either a prominent or attenuated frequency; deriving a first audio signal including the exceptional frequency; measuring one or more physiological characteristics indicative of a patient breathing rate and of a patient stress level; deriving a second audio signal from the patent breathing rate, wherein the second audio signal is a repeated and/or spatially oscillating at a second audio frequency no greater than the patient breathing rate; and playing the first and second audio signals to the patient for a period of a treatment session, wherein the first and second audio signals are played simultaneously for at least a portion of the treatment session.

### CROSS REFERENCES TO RELATED APPLICATIONS

This application is a U.S. national phase entry of, and claims priority to, PCT International Patent Application No. PCT/IL2021/051163, which claims priority to U.S. Provisional Patent Application No. 63/082,831, filed Sep. 24, 2020. The entire contents of the above-referenced applications and of all priority documents referenced in the Application Data Sheet filed herewith are hereby incorporated by reference for all purposes.

### FIELD OF THE INVENTION

The present invention relates to the field of patient stress treatment, and more particularly, to non-invasive, biofeedback treatments.

### BACKGROUND

Stress and stress-related diseases, such as hypertension, anxiety, indigestion, and sleep disorders, are common problems that are difficult to treat. Various health promoting methods are described in U.S. Patent Application Publication Nos. 2017/202509 and 2008/208015 and in U.S. Pat. Nos. 8,784,311 and 10,561,361, all of which incorporated herein by reference in their entirety.

### SUMMARY

Embodiments of the present invention provide a system and methods for patient treatment, including steps of: receiving sounds vocalized by a patient; determining, from the vocalized sounds, an exceptional frequency that is either a prominent or attenuated frequency; deriving a first audio signal including the exceptional frequency; measuring one or more physiological characteristics indicative of a patient breathing rate and of a patient stress level; deriving a second audio signal from the patient breathing rate, wherein the second audio signal is a repeated and/or spatially oscillating at a second audio frequency no greater than the patient breathing rate; and playing the first and second audio signals to the patient for a period of a treatment session, wherein the first and second audio signals are played simultaneously for at least a portion of the treatment session. The exceptional energy level may be identified by frequency analysis of the patient's speech.

In some embodiments, the second audio frequency is slower than the patient breathing rate or is slowed during the treatment session to a rate that is slower than the patient breathing rate. The first audio signal may be a human breathing sound, and it may be a binaural beat created from two tones, where the two tones have frequencies separated by a gap that is a transposition of the exceptional sound frequency. The gap may be in the range of 0.1 to 30 Hz, and a mean of the two tones may be set to the exceptional sound frequency. The system of claim 1, wherein the first and second audio signals are played at a volume dependent on the patient stress level, and wherein the volume is increased during the treatment session as the patient stress level drops.

Playing the first and second audio signals to the patient is typically implemented by playing the audio signals through headphones of the patient. One of the two audio signals may be played at the start of the treatment session, then the two audio signals may be played simultaneously for a second period of the treatment session. The first or second audio signal may then be played by itself for a third period of the treatment session.

In some embodiments, a third audio signal may be playing simultaneously with the first and second audio signals during at least a portion of the treatment session. The third audio signal may include binaural 3D nature sounds. Alternatively, the third audio signal may be the exceptional energy sound frequency. The third audio signal may be spatially varying with an oscillation corresponding to a rate that is similar or lower that a frequency of a monitored heart rate variability parameter, an EEG signal parameter, or the breathing rate of the patient.

In some embodiments, the system further includes characterizing a responsiveness of the patient's auditory, auricular trigeminal and/or vagus nerves to the audio signals and responsively adjusting a frequency and/or volume of the audio signals.

In some embodiments, the system further includes delivering to the patient tactile and/or visual stimulation during the treatment session.

In some embodiments, the system further includes adjusting a volume of the audio signals to the patient's schedule and environment.

In some embodiments, the system further includes analyzing accumulated data from multiple patients to enhance the derivation of the audio signals.

In some embodiments, the system further includes providing a user interface for presenting bio-feedback, wherein the user interface includes visual, gaming and/or social network features.

In some embodiments, the system further includes implementing bio-resonance techniques to measure energy frequencies of the patient and using them in diagnosis and/or treatment.

In some embodiments, the system further includes implementing eye movement desensitization and reprocessing (EMDR) procedures of eye movement monitoring during the treatment session.

The measured physiological characteristics may also include EEG signals.

### BRIEF DESCRIPTION OF DRAWINGS

For a better understanding of various embodiments of the invention and to show how the same may be carried into effect, reference will now be made, by way of example, to the accompanying drawings. Structural details of the invention are shown to provide a fundamental understanding of the invention, the description, taken with the drawings, making apparent to those skilled in the art how the several forms of the invention may be embodied in practice. In the accompanying drawings:

FIG. 1 is a schematic block diagram of a system for patient treatment, according to some embodiments of the invention;

FIG. 2 is a schematic example of audio signals applied by the system, according to some embodiments of the invention; and

FIG. 3 is a flowchart illustrating a method for patient treatment, according to some embodiments of the invention.

### DETAILED DESCRIPTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

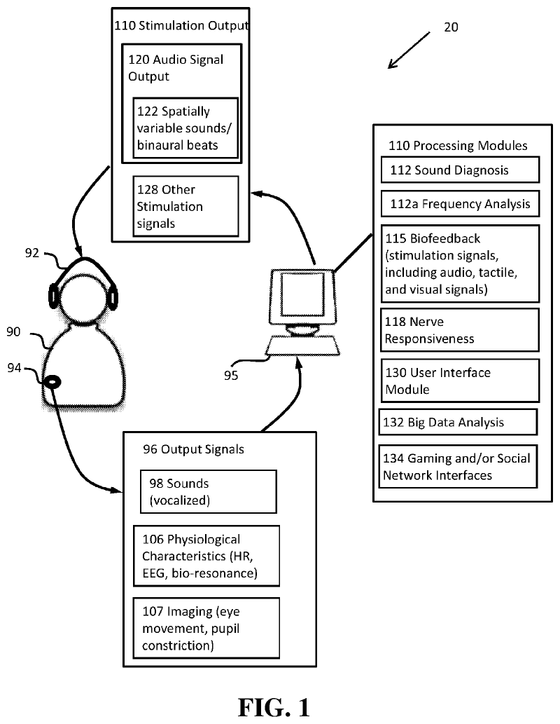

FIG. 1 is a high-level schematic block diagram of a system 20, according to some embodiments of the invention. System 20 may be applied to treat stress in patients 90 including treatment of sleep disorders, providing a non-invasive, patient-specific, audio-based biofeedback procedure. System 20 and/or its processing modules may be implemented by a computing device 95 having the disclosed inputs and output, and/or as software modules 110 that may be run on specialized and/or generalized hardware such as processors (for example, in computing devices such as computers, handheld devices, communication devices such as smartphones, etc.), speakers and/or headphones 92, as disclosed herein. In some embodiments, system 20 and/or its processing modules may be at least partly implemented at a remote computing environment such as cloud computers, cloud servers and/or cloud network, and be linked to the patient's hardware via one or more communication links.

One or more sensors 94 provide output signals 96 to processing modules 110. Sensors may include microphones that pick up sounds 98 vocalized by the patient. Physiological characteristics 106 may be measured, for example, by sensors including generic pulse and breathing measurement devices (e.g., smartwatch or fitness appliances, and/or bio-resonance electrodes or galvanic measurement devices). In certain embodiments, system 20 may be configured to measure imaging output 107, for example, using imaging sensors such as may be provided by a smartphone. Pupil parameters may be measured optically as well, using, for example, imaging device(s), eye tracker(s), smart glasses, etc. Pupil parameters may include pupil size that may be used to indicate the activity of the autonomic nervous system (ANS) and provide biofeedback data with respect to nerve stimulation (especially with respect to the vagus nerve stimulation, as described below). Eye movements and/or pupil parameters may be measured before, during and/or after the treatment. Eye movements and/or pupil parameters may also be measured, using generic eye tracking devices, image analysis and/or smart glasses, and be related to ANS activity. In certain embodiments, system 20 may be configured to implement eye movement desensitization and reprocessing (EMDR) procedures, in association with generation of spatially varying sounds, providing eye movement monitoring and biofeedback treatment to alleviate stress, distressing thoughts, trauma symptoms etc. For example, in addition to an EMDR technique of asking the patient to follow moving objects, disclosed embodiments may enhance EMDR procedures by adding, for example, spatially varying sounds. Hereinbelow, spatially varying sounds are sounds that a patient perceives as “moving,” either from side to side due to changing amplitudes of stereo components of the audio signal, or moving in the full 3D space around the patient by means of binaural recording and playback of binaural audio signals. Spatially varying sounds may be used as auditory stimuli to support and enhance EMDR procedures, for example to cause specific eye movements.

Processing modules 112 may include sound-based diagnosis 112 applied to the patient's speech. The diagnosis may identify attenuated and/or prominent features in the patient's speech, such as specific vowels or consonants that are over- or under-expressed. The diagnosis may also identify specific sound frequencies that are over- or under-expressed. The patient's speech may comprise free speech or guided speech, for example, in conversation, reading specific texts (for example having specified lengths and specified durations dedicated for the reading), in Karaoke mode with an accompaniment, or using other methods. In certain embodiments, system 20 may be configured to perform sound-based diagnosis 112 of arbitrary sounds, words and/or sentences produced by patient 90, for example, in response to various stimuli or instructions, or freely.

In some embodiments, system 20 may be configured to apply a frequency analysis 112A of the patient's sounds and/or speech (for example, using a fast Fourier transform applied to the recorded signals) to identify attenuated and/or prominent sound frequencies in the patient's speech or produced sounds. (Attenuated frequencies may also include missing frequencies.) In some embodiments, frequency analysis 112A may also be used to derive breathing and/or heartbeat related signals by analyzing the user's produced sounds, and use related parameters as part of sound-based diagnosis 112. Frequency analysis 112A may thus complement, enhance or replace the measurement of physiological parameters 106.

Processing modules 110 may be further configured to measure physiological characteristics 106 of patient 90 that comprise at least one of heart rate variability (HRV), pulse rate, bio-resonance signals, pupil parameters and/or breathing parameters, before, during and/or after the treatment of patient 90 by system 20. Measurement of physiological characteristics 106 may be carried out continuously or intermittently. In certain embodiments, system 20 may be configured to measure EEG signals (or EEG-like signals) as part of physiological characteristics 106, for example, via the physical contact regions of headphones 92 with patient 90, or via other sensors 94 in contact with the patient's body (such as an EEG sensors associated with headphones 92), or remotely. The EEG signals or EEG-like signals may likewise be used as feedback parameters with respect to ANS stimulation. In various embodiments, spatially varying binaural beats, other spatially varying sounds 122 and/or other types of sounds described below may be configured to have a perceived oscillating movement (which may also be rotating around the patient) at a similar or lower frequency than parameters of measured EEG signals of patient 90.

In certain embodiments, system 20 may be configured to receive additional patient's input, for example, using questionnaires.

Processing modules 110 may be further configured to derive by biofeedback 115, from the sound-based diagnosis 112 and from measured physiological characteristics 106, audio signals 120 that may include spatially varying sounds or tones, repetitive sounds or tones, and/or binaural beats 122, as well as other various types of noise, including synthetic breathing and/or heartbeats, as well as nerve stimulation signals. Audio signals 120 are patient-specific and selected to implement stress relief and/or treat sleep disorders in patient 90. In any of the disclosed embodiments, nerve stimulation signals may be separately added to audio signals 120 and/or may be part of audio signals 120. For example, frequencies of components of audio signals 120 may be selected to stimulate specific patient's nerves, such as vagus nerves passing in the ear region. Audio signals 120 may be adjusted to provide patient-specific nerve stimulation, for example, in relation to the patient's ear region and nerve anatomy. Processing modules 110 may be further configured to deliver audio signals 120 to patient 90 as biofeedback while monitoring measured physiological characteristics 106.

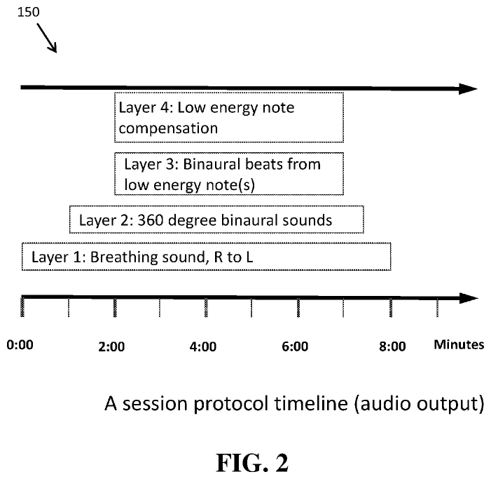

In certain embodiments, system 20 may be configured to derive audio signals 120 with respect to the identified attenuated and/or prominent features in the patient's speech or produced sounds, such as missing or low energy frequencies, or excessive or high energy frequencies in the patient's speech or vocalized sounds (low or high energy frequencies also being referred to hereinbelow as exceptional energy frequencies). In certain embodiments, audio signals 120 may be derived to alternate between provision of compensating features and intermittent relaxing sounds or music, specific recorded words and/or sounds in specified treatment frequencies. Alternatively or additionally, multiple types of audio signals 120 may be delivered simultaneously, possibly in different perceived spatial regions (see, for example, audio protocol shown in FIG. 2). In certain embodiments, instructions concerning breathing may be incorporated with delivered audio signals 120 and/or as part of the biofeedback procedures. Audio signals 120 may be generated to correspond to brainwave frequencies, such as theta waves within 4-7 Hz, alpha waves within 7-5 Hz, and Schumann resonance frequencies of 7.8 Hz and harmonies thereof, possibly with daily updates to values.

In certain embodiments, system 20 may be configured to implement bio-resonance techniques to measure energy frequencies of the patient and using them in diagnosis and/or treatment. In certain embodiments, system 20 may be configured to implement grounding (or earthing) techniques (electrically grounding the patient to the earth, to control the exchange of electric charges to and from the patient) to achieve positive effects on the patient such as soothing and alleviating stress.

In various embodiments, audio signals 120 may comprise any of: binaural beats (which may be spatially varying), breathing sounds, various types of sounds (spatially varying sounds or tones, repetitive sounds or tones, various noise types such as white noise), nerve-simulating sounds, verbal signals (words, sentences, syllables etc.), music notes or sounds using various playback techniques. In various embodiments, system 20 may be configured to derive and deliver to the patient stimulation signals 128 in addition to audio signals 120. Non-limiting examples for stimulation signals include tactile stimulation (for example, vibrations delivered to the patient's skin, ear(s), scalp, etc.), and visual stimulation (e.g., specific images, light, colors, illumination and/or color pulses for nerve stimulation, etc.) and/or verbal stimulation (e.g., instructions to produce specific sounds or tones, read certain words or sentences, etc.). Any of the stimulation signals 128 may be derived according to sound-based diagnosis 112 and/or measured physiological characteristics 106. Any of the stimulation signals 128 may be delivered in coordination with audio signals 120 to enhance the effects thereof. The selection and combination of various audio signals 120 and stimulation signals 128 during one or more treatment may be carried out with respect to diagnostic features relating to the patient and/or with respect to data accumulating concerning multiple patients and treatment effectiveness thereof. In certain embodiments, one or more types of audio signals 120 and/or of stimulation signals 128 may be selected according to the monitored HRV, measured EEG signal parameters and/or a breathing rate of the patient.

In certain embodiments, system 20 may be further configured to characterize a responsiveness of the patient's auditory, auricular trigeminal and/or vagus nerves to auditory excitation, for example, via a nerve responsiveness diagnosis module 118 and adjust the nerve stimulation respectively. One or more of the nerves may be stimulated at a time. For example, the nerve responsiveness diagnosis 118 may relate a varying acoustic stimulus to a patient's reaction, as measured by changes in physiological characteristics 106 such as the HRV, for example, in a frequency scanning (of a specified acoustic range within a specified time period. Audio frequency scanning may be carried out automatically within specified range(s) (e.g., within 1-20 Hz or sub-ranges thereof, and 80-90 Hz, or within other ranges) and during a specified period (e.g., one or two minutes, or other durations). During the audio frequency scanning, respective nerves such as the vagus nerve may be measured to identify their responses to the audio stimulation, to derive therefrom an optimal nerve stimulation frequency or frequencies. In certain embodiments, audio frequency scanning may also be implemented in an adjustment procedure as part of the biofeedback process. Nerve responsiveness may be further measured spatially, to identify the optimal locations around the patient's ears to apply the acoustic nerve stimulation. Specific acoustic nerve stimulation with respect to different nerves, frequencies and locations may be applied as part of the audio signal delivery, in relation to and/or independent from the delivery of spatially varying binaural beats 122. During the treatment, nerve stimulation frequencies may be adjusted with respect to the patient's responses in measured physiological characteristics 106 or otherwise. In any of the disclosed embodiments, nerve stimulation may comprise excitation and/or attenuation of various nerves, for example vagus excitation and trigeminal attenuation, possibly simultaneously, alternatingly or in any other combination, as well as the opposite stimulation types of either of the nerves.

The inventors have found that non-invasive sounds or other pressure types applied to the patient's ears, in particular at specified frequencies, may stimulate various nerves and contribute to relaxation and treatment. Affected nerves may include the afferent auricular branches of the vagus nerve (aVN) and regions of auriculotemporal branch of the trigeminal nerve and of the great auricular nerve.

For example, sound frequencies selected to stimulate nerves via vibrations to the respective nerves may be used to activate the nerves themselves. The stimulation signals may be applied via the headphones used by the patient. In certain embodiments, stimulation signals may be adjusted to the patient's specific nerve anatomy by adjusting the location of their application and their frequencies, for example, utilizing geometrical considerations.

The biofeedback module 115 associated with processing module 110 may be configured to modify audio signals 120 according to reactions of patient 90, such as changes in patient's physiological characteristics 106 and/or other patient reactions. Biofeedback may be implemented in various ways. For example, an audio signal may be generated that simulates the sound of human breathing, with the rate of simulated breathing modified according to changes in the patient's actual breathing rate.

Audio signals 120 may be derived to compensate for inaccuracies in various sounds within the patient's range. Biofeedback module 115 may be configured to provide online (real-time) biofeedback tasks and/or offline (training) tasks.

In certain embodiments, visual and/or tactile feedback stimulation signals 128 may be provided in addition to audio feedback, for example, a reduction in illumination intensity and/or in tactile signals (e.g., vibrations) may accompany a reduction in audio frequencies or in perceived audio motion frequency. Visual and/or tactile feedback may be delivered via a dedicated and/or via a generic user interfaces such as the patient's smartphone, smart glasses and/or via elements associated with headphones 92 (and/or corresponding speakers or transducers). Visual feedback may be delivered, possibly in relation to audio signals 120, for example specific colors and/or intensities as well as pulses and/or changes thereof, or specific images—may be presented with respect to specific audio signals 120, and biofeedback may be provided at least partly with respect to the patient's reactions to the visual stimuli.

In certain embodiments, system 20 may be further configured to analyze accumulated data from multiple patients to enhance the derivation of the audio signals, for example, implementing big data analysis 132 to derive new patterns and relations between delivered audio signals 120 and patient relaxation and/or treatment of sleep disorders, cognitive disorders, somatic complaints, physical symptoms and/or issues related to the patient's homeostasis. Artificial intelligence procedures may be implemented to derive such new patterns and relations from data accumulated from many treatment sessions, and thereby improve the efficiency of disclosed systems 20 over time. For example, new relations between parameters of spatially varying binaural beats/sounds 122 and nerve stimulation and the treatment efficiency of various conditions may be deciphered using big data analysis and implemented in consecutive treatment procedures.

In certain embodiments, system 20 may comprise a user interface module 130 for interaction with patient 90. The user interface may also be associated with a gaming platform 134, incorporating disclosed biofeedback mechanisms within a game played by patient 90. Audio signals 120 may be configured to be part of the respective game, and/or patient relaxation parameters may be made part of the game to increase patient motivation and treatment efficiency. For example, increased relaxation may be rewarded in the game, for example, in relation to parameters of the treatment such as patient's physiological characteristics 106 and/or audio signals 120. In certain embodiments, spiritual practices and/or relaxation techniques may be combined with the acoustic biofeedback and/or in the gaming platform.

In certain embodiments, system 20 may comprise user interface 130 which is associated with a social networking platform 134, incorporating disclosed biofeedback mechanisms within the interactions of patient 90 in the social network. Audio signals 120 may be configured to be part of the respective social interaction, and/or patient relaxation parameters may be made available over the social network to increase patient motivation and treatment efficiency. For example, increased relaxation may be rewarded in the social network, for example, in relation to parameters of the treatment such as patient's physiological characteristics 106 and/or audio signals 120. Gaming and social networking 134 may be combined in relation to disclosed biofeedback mechanisms to enhance treatment efficiency. Social networking may include a dating platform, incorporating the biofeedback mechanisms disclosed herein within the interactions of patient 90 with possible partners and to estimated matching of the patient with possible partners. Audio signals 120 may be configured to be part of the respective date selection and dating interaction, and/or patient relaxation parameters may be made available over the dating platform to increase matching success as well as patient motivation and treatment efficiency. For example, partners may be matched with respect to their identifying attenuated and/or prominent features in the patient's speech or produced sounds (for example, as having matching and/or complementary parameters), with respect to their nerve responsiveness, with respect to the patient's brain activity patterns and/or in relation to other information provided by the dating platform. Gaming, social networking and dating platform 134 may be combined in relation to disclosed biofeedback mechanisms to enhance treatment efficiency.

System 20 may be further configured to incorporate follow-up procedures to measure the patient's stress and/or sleep disorders over time (possibly during several treatment sessions), assess the efficiency of the biofeedback treatment and possibly improve the applied procedures to optimize treatment. For example, various cognitive assays and medical diagnosis procedures may be used to assess treatment efficiency.

FIG. 2 is a schematic example of a protocol 150 for generation of audio signals 120, according to some embodiments of the invention. Any of disclosed audio signals 120 may be applied to the patient (i.e., transmitted to acoustic transducers, that is, audio speakers such as headphones 92) in various “temporal patterns,” that is, during various periods of a treatment session. As indicated, audio signals 120 may comprise multiple sound layers, which may be added or removed from a timeline of a session according to specified protocols, customized for patient characteristics and real-time environmental parameters. As described below, audio layers may include binaural beats, spatially varying sounds or tones, breathing or heartbeat sounds, various types of noise or synthetic sounds or tones, etc.

Notes may be added to, or removed from audio signals 120 according to the strength of their vocalization by the patient. Additionally, stimulation signals 128 of various types may be introduced along the same timeline of a session protocol to enhance the treatment and the biofeedback of audio signals 120. Any of the audio signal layers may be added or removed, or relevant parameters thereof (e.g., frequencies, rates, intensity, etc.) may be adjusted at any time during the treatment (and between different treatments), for example, by the treating personnel or as response to the biofeedback parameters or patient's input.

As described above, system 20 may be configured to analyze the patient's vocalized range of sounds. Audio signals 120 may include audio layers that are derived to treat separately sub-ranges of the patient's total range, possibly in terms of musical notes and/or intervals within the overall range.

In further embodiments, audio signals 120 may comprise breathing sounds at a rate that is the same or lower than a monitored patient's breathing frequency. For example, a decreasing rate of breathing sounds may be used to relax patient 90. The breathing sounds may be recorded (from patient 90 or not) or be synthetic breathing and/or heartbeat sounds that may be produced using algorithms and/or electronic circuitry (e.g., digital or analog oscillator(s), low frequency oscillator(s), etc.) that may require a smaller storage volume than pre-recorded sounds. Any of audio signals 120 may be pre-recorded or generated synthetically (e.g., using various basic signals such as sine or triangular waveforms) to reduce storage requirements and enhance real-time responsiveness of system 20.

In certain embodiments, audio signals 120 may be adjusted to the patient's schedule and environment, for example, audio signals 120 may be louder when patient 90 is in a loud environment and softer when patient 90 is in a quiet environment, and/or the intensity of audio signals 120 may be adjusted to the patient's physiological cycles, such as the patient's circadian rhythm and/or to the patient's current levels of stress, anxiety, sleeplessness, etc.

Binaural beats 122 are paired audio tones that have frequencies that are close to each other and thus cause a perceived beating sound at the frequency of the gap between the pair. Frequency gaps ranging from 0.1 Hz to 30 Hz be synchronized to brainwave frequencies in order to enhance or attenuate specific brainwave patterns, contributing to relaxation. For example, system 20 may be configured to implement brainwave entrainment to contribute to stress relief. Spatially varying binaural beats and/or other spatially varying sounds or tones 122 may be configured to change in the perceived spatial location of the beating audio signal, to form a perceived motion of the beating audio signal. In various embodiments, spatially varying binaural beats and/or other spatially varying sounds 122 may be configured to have a perceived spatially oscillating movement (i.e., back-and-forth motion, which may also be rotating around the patient). A decreasing rate of oscillation of the spatially varying sounds 122 may be used to relax patient 90. Alternatively or complementarily, the repetition frequency of repetitive sounds may be modified in a similar manner. In some embodiments, the perceived location, repetition frequency or movements of audio signals 120 may be configured to treat disorders, such as muscular tensions, cognitive disturbances, and digestive problems. Cognitive improvements may include improvements in memory, concentration, learning ability, etc. Additional patient input 107 may be used to determine treatments, and be used to adjust the perceived location or movements of spatially varying binaural beats/sounds 122 respectively. In certain embodiments, biofeedback may be implemented using spatial relations between spatially varying binaural beats/sounds 122 and patient movements, such as hand movements, eye movements, pupil dilation etc.

In certain embodiments, decay durations and/or pitches of binaural beats 122 may be used to enhance or partly replace perceived motion rates thereof. In certain embodiments, perceived spatial locations of binaural beats/sounds 122 may be used to provide biofeedback to patient 90, for example, patient 90 may be encouraged to cause certain perceived locations to change into other locations as biofeedback, by modifying the patient's physiological characteristics 106. Any of these or other perceived parameters of binaural beats or sounds 122 may be modified with respect to any of the patient's physiological characteristics 106 (e.g., breathing rate, heartbeat rate) to provide the biofeedback.

In certain embodiments, audio signals 120 may be configured to directly provide nerve stimulation, for example, audio signals 120 may be derived to stimulate the auditory nerve, the auricular trigeminal nerve and/or the vagus nerve. Audio signals 120 may be configured to deliver non-invasive nerve stimulation, via the pressure waves emanating from headphones 92 or possibly through auxiliary pressure-applying elements. In certain embodiments, the perceived beating frequency of binaural beats 122 or of other repetitive sounds may be adjusted to provide nerve stimulation. Various stimulation patterns may be implemented to convey relaxation via nerve stimulation.

Protocol 150 includes four audio signal layers, layers 1-4, which may be played to the patient at various overlapping periods during a treatment session. A length of a treatment session is typically set by a patient and may vary depending on factors such as a patient's goals and environment. A “sleep mode” treatment session may, for example, be set to 40 minutes. The treatment shown in protocol 150 is shown as being 8 minutes.

Each layer is derived by a method incorporating a different aspect of the audio signal generation methods described above. For example, layer 1, which is played from the starting time of a treatment session (0:00) until the end of the session may be derived from the patient's measured breathing rate. The breathing rate, as described above, may be derived from a direct measurement or by an estimation based on other measured physiological characteristics, such as the heart rate. The generated audio signal of layer 1 may be a sound of human breathing, repeated at a rate that is slower than the breathing rate of the patient, for example, 2%-10% slower, or which gradually slows to this level. The slower rate of the simulated sound causes a calming effect on the patient. Slowing the generated audio signal rate from an initial rate set to the breathing rate further promotes such calming.

In addition, the breathing sound of layer 1 may generated as a spatially varying sound, that is, a sound that the patient perceives as moving back and forth from one side of the patient to the other, also promoting a reduction in stress.

Additional audio layers may be played simultaneously with layer 1, with or without an initial delay. For example, a layer 2 may be introduced after 1 minute. Layer 2 may be a calming background sound, such as a 3D binaural sound of nature, such as a 3D binaural sound generated in a forest. Such nature sounds allow the user to experience a calming environment that reduces distractions from inner and outer disturbances during the session.

Additional audio layers may be generated, for example, from a measurement of an exceptional energy frequency vocalized by the patient (either prominent or attenuated energy). A low energy frequency, for example, that is, a frequency generated by the patient's vocal cords at a weaker level than other frequencies of a given musical octave, may be used as a carrier frequency of a binaural beat that stimulates the vagus nerve. Such a binaural beat may be generated as an audio layer 3. A binaural gap for such a layer may be calculated by dividing the low energy frequency by multiples of 2 until reaching a relatively low frequency, such as a frequency in the Delta range of 0.1-4 Hz, or at most a beat of 30 Hz.

For example, for a low energy frequency of 256 Hz (slightly below the note “middle C”) calculation of the gap would be: 256/2=128, 128/2=64, 64/2=32, 32/2=16, 16/2=8, 8/2=4 (i.e., transposing the note by 6 octaves). Consequently, we can apply a gap of 4 Hz to the carrier frequency of 256. The two tones that would make the binaural beat would thus be: 256−2=254 Hz and 256+2=258 Hz.

A fourth audio layer (layer 4) may be an audio signal set at the low energy frequency, determined in the manner described above. The audio signal may also be set to the equivalent musical note represented by the low energy frequency but transposed to a lower octave, to further stimulate the autonomic nervous system (ANS), by stimulating the vagus nerve branch (which also innervates the vocal cords). Audio layer 4 may also be configured as a binaural sound that the patient may perceive as rotating around his body.

The low energy frequency applied in generating audio layers described above may also be calculated from a high energy frequency determined from sounds vocalized by the patient. For example, if a musical octave is set to start at a frequency vocalized at a relatively high energy level, the middle note of the octave (the “augmented fifth,” which has a frequency of the base note multiplied by the square root of two) would be considered the low energy frequency. Alternatively, the high energy frequency may be used as the mean of the binaural beat and as the base frequency for calculating the gap frequency (by transposition).

As indicated by protocol 150, layer 2 may be played to the patient starting, for example, after a 1 minute delay from the start of a session and may end half a minute before the end of the session. Layer 3 may begin, for example, two minutes after the start of the session and end a full minute before the end of the session. Layer 4 may begin 3 minutes after the start of the session and also end a minute before the end of the session.

During the course of the session, the system may continue to measure physiological characteristics of the patient. If the patient's breath rate declines, the simulated breathing rate of the analog signal of layer 1 may be similarly reduced to encourage further stress reduction.

In addition, any of the measured physiological characteristics, measured or derived as described above, that are indicative of stress may be applied to determine a stress index. For example, heart rate variability (HRV) may be used as an indicator of stress, with lower HRV indicative of a higher stress level. (See, for example, Shaffer and Ginsberg, “An Overview of Heart Rate Variability Metrics and Norms,” Frontiers in Public Health, 2017.

Measures of HRV may be: root mean square of successive differences (rMSSD), standard deviation of the normal-to-normal interval (SDNN), and HRV spectral components, e.g., the high-frequency band (HF), or low-frequency band (LF). A range of several stress levels, such as five levels ranging from high to low, can be used for biofeedback, whereby the volume of the audio layers is adjusted according to the stress level, in an inverse manner (lower volume when higher stress measured, and vice versa), until an optimal stress level is reached. (An optimal stress level may be indicated, for example, by a flattening of an HRV curve, or even a subsequent HRV decline.) Volume levels may be set to vary from a level of whispering (approximately 30 dB) to a loud conversational level (approximately 70 dB) as stress levels decline.

It is to be understood that a session may be conducted with any one of the above layers, played alone or in conjunction with any other one or more layers, in order to achieve a reduced level of patient stress. Embodiments of the present invention may include determining from vocalized patient sounds an exceptional energy frequency of sound, the exceptional frequency being either a high or low energy frequency among frequencies of the vocalized sound;

measuring at least one physiological characteristics of the patient including at least one of heart rate variability (HRV), a pupil parameter, and a breathing rate; deriving audio signals from the sounds and from the measured physiological characteristics, including at least one of spatially varying sounds, based on the breathing rate, and binaural beats, based on the exceptional energy frequency; playing the audio signals to the patient at a volume dependent on the at least one physiological characteristic; and adjusting the volume according to changes in the at least one physiological characteristic measured while playing the audio signals to the patient. The system may also provide the patient with visual feedback indicative of the patient's stress level.

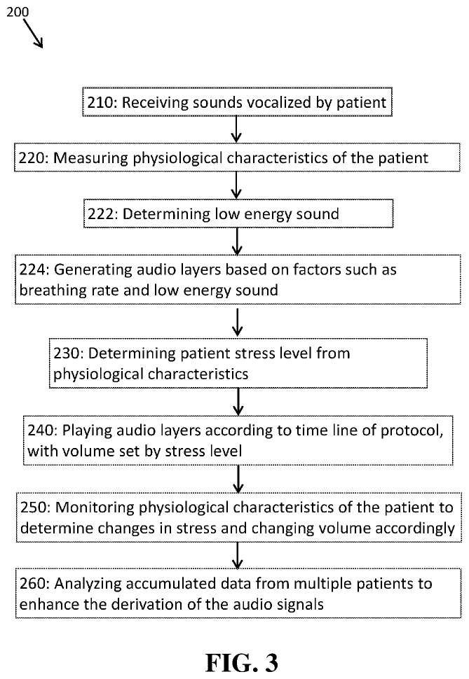

FIG. 3 is a flowchart illustrating a method 200, according to some embodiments of the invention. The method stages may be carried out with respect to system 20 described above, which may optionally be configured to implement method 200. Method 200 may be at least partially implemented by at least one computer processor, such as the computing device 95, which may be, for example, a personal computer, a hand-held device, or smartphone. Certain embodiments comprise computer program products comprising a computer readable storage medium having computer readable program embodied therewith and configured to carry out the relevant stages of method 200. Method 200 may comprise the following stages, irrespective of their order.

Method 200 may comprise recording and analyzing sounds produced by the patient (stage 210) and measuring physiological characteristics of the patient (stage 220). As described above, physiological characteristics may include at least one of a heart pulse rate (i.e., heartbeat), heart rate variability (HRV), eye movement, pupil parameters (e.g., pupil constriction), breathing rate, EEG signals, and bio-resonance signals. Subsequently the method includes deriving, from the sound-based diagnosis, a low energy frequency (stage 222).

Next, the measured physiological characteristics and the low energy frequency are applied, as described above, to calculate signals that will be generated as one or more audio layers (stage 224). Such layers may include at least one of: spatially varying sounds or tones, repetitive sounds or tones, and binaural beats. Layers include audio nerve stimulation signals, as described above. In addition, before playing the audio layers, the measured physiological characteristics may be processed to determine a stress level of the patient (stage 230), which can be applied to modify attributes of the audio layers, in particular, the volume, as described above. The audio layers may then be played accorded to a predetermined protocol (stage 240). The audio layers are provided to the patient (e.g., transmitted to the patient headphones) while physiological characteristics continue to be monitored, thereby providing biofeedback to the system, which may in turn change the signals of the audio layers (stage 250). Audio changes due to the biofeedback may include, for example, the volume and the rate of sound repetition. The user interface may also provide the patient with a real-time, visual indication of the patient's stress level.

Method 200 may further comprise analyzing accumulated data from multiple patients to enhance the derivation of the audio signals (stage 260).

In certain embodiments, method 200 may further comprise implementing bio-resonance techniques to measure energy frequencies of the patient and using the frequencies in diagnosis and/or treatment. Method 200 may further comprise implementing eye movement desensitization and reprocessing (EMDR) procedures in association with eye movement monitoring, biofeedback treatment and/or in association with changes in the spatially varying sounds, to alleviate stress. Method 200 may further comprise delivering to the patient tactile and/or visual stimulation derived according to the sound-based diagnosis and/or the measured physiological characteristics.

The sounds vocalized by the patient may include speech, and analyzing 210 may comprise identifying attenuated and/or prominent features in the patient's speech. For example, the attenuated and/or prominent features in the patient's speech may be identified by frequency analysis of the patient's speech, and the audio signals may be adjusted with respect to the identified attenuated and/or prominent features in the patient's speech.

In certain embodiments, the spatially varying binaural beats and/or sounds may be configured to spatially oscillate (which may include rotation) at a similar or lower frequency than one of the following: the monitored HRV, pulse rate, bio-resonance signals, parameters of EEG signals, and/or breathing rate of the patient. Alternatively or complementarily, the repetition frequency of repetitive sounds (such as breathing sounds) may be modified in a similar manner.

In certain embodiments, the nerve stimulation signals may comprise audio signals and/or other pressure signals configured to stimulate the auditory nerve, the auricular trigeminal nerve and/or the vagus nerve. In certain embodiments, method 200 may further comprise characterizing a responsiveness of the patient's auditory, auricular trigeminal and/or vagus nerves to auditory excitation and adjusting the nerve stimulation respectively.

Method 200 may further comprise selecting at least one of the audio signals and/or the stimulation signals according to the monitored HRV, measured EEG signal parameters and/or a breathing rate of the patient. Method 200 may further comprise adjusting the audio signals to the patient's schedule and environment.

In certain embodiments, the audio signals may further comprise synthetic breathing sounds and/or heartbeat sounds at a rate same or lower than a monitored patient's breathing frequency.

In certain embodiments, method 200 may further comprise providing a user interface for the biofeedback, which includes visual, gaming and/or social network features (stage 240).

Exemplary computing device 95, which may be used with embodiments of the present invention may include a controller or processor that may be or may include, for example, one or more central processing unit processor(s) (CPU), one or more Graphics Processing Unit(s) (GPU or general-purpose GPU-GPGPU), a chip or any suitable computing or computational device, an operating system, memory and non-transient memory storage including instructions, input devices and output devices. Processing steps of system 20, including processing module 110 and/or biofeedback module 115, big data analysis module 132, gaming and/or social networks module 134, and user interface 130, operating online and/or offline, may be executed by computing device 95. In various embodiments, computing device 95 may comprise any of the devices mentioned above, including for example, communication devices (e.g., smartphones), visibility enhancing devices (e.g., smart glasses), various cellular devices with recording and playback features, optical measurement and imaging devices, cloud-based processors, etc.

The operating system may be or may include any code segment designed and/or configured to perform tasks involving coordination, scheduling, arbitration, supervising, controlling or otherwise managing operation of computing device 95, for example, scheduling execution of programs. Memory may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory may be or may include a plurality of possibly different memory units. Memory may store for example, instructions to carry out a method, and/or data such as user responses, interruptions, etc.

Instructions may be any executable code, for example, an application, a program, a process, task or script. Executable code may be executed by possibly under control of the operating system of the computing device. For example, executable code may when executed cause the production or compilation of computer code, or application execution such as VR execution or inference, according to embodiments of the present invention. Executable code may be code produced by methods described herein. For the various modules and functions described herein, one or more computing devices 95 or components of computing device 95 may be used. Devices that include components similar or different to those included in computing device 95 may be used, and may be connected to a network and used as a system. One or more processor(s) may be configured to carry out embodiments of the present invention by for example executing software or code, and may act as modules and computing devices described herein.

Non-transient memory storage may be or may include, for example, a hard disk drive, a floppy disk drive, a Compact Disk (CD) drive, a CD-Recordable (CD-R) drive, a universal serial bus (USB) device or other suitable removable and/or fixed storage unit. Data such as instructions, code, model data, parameters, etc. may be stored in a storage and may be loaded from storage into a memory where it may be processed by controller.

Input devices may be or may include for example a mouse, a keyboard, a touch screen or pad or any suitable input device. It will be recognized that any suitable number of input devices may be operatively connected to computing device 95. Output devices may include one or more displays, speakers and/or any other suitable output devices. It will be recognized that any suitable number of output devices may be operatively connected to computing device 95. Any applicable input/output (I/O) devices may be connected to computing device, for example, a wired or wireless network interface card (NIC), a modem, printer or facsimile machine, a universal serial bus (USB) device or external hard drive may be included in input devices and/or output devices.

Embodiments of the invention may include one or more article(s) (e.g., memory or storage) such as a computer or processor non-transitory readable medium, or a computer or processor non-transitory storage medium, such as for example a memory, a disk drive, or a USB flash memory, encoding, including or storing instructions, for example, computer-executable instructions, which, when executed by a processor or controller, carry out methods disclosed herein, or configure the processor to carry out such methods.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions.

These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram or portions thereof.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams may represent a module, segment, or portion of code, which comprises one or more executable instructions for implementing the specified logical function(s). It should also be noted that, in some alternative implementations, the functions noted in the portion may occur out of the order noted in the figures. For example, two portions shown in succession may, in fact, be executed substantially concurrently, or the portions may sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Method steps associated with the system and process can be rearranged and/or one or more such steps can be omitted to achieve the same, or similar, results to those described herein. It is to be understood that the embodiments described hereinabove are cited by way of example, and that the present invention is not limited to what has been particularly shown and described hereinabove. Rather, the scope of the present invention includes variations and modifications thereof which would occur to persons skilled in the art upon reading the foregoing description and which are not disclosed in the prior art.

1. A system for patient treatment, comprising a processor having associated non-transient memory including instructions that when executed cause the processor to perform steps of:
1) receiving sounds vocalized by a patient;
2) determining, from the vocalized sounds, an exceptional frequency that is either a prominent or attenuated frequency;
3) deriving a first audio signal including the exceptional frequency;
4) measuring one or more physiological characteristics indicative of a patient breathing rate and of a patient stress level;
5) deriving a second audio signal from the patient breathing rate, wherein the second audio signal is a repeated and/or spatially oscillating at a second audio frequency no greater than the patient breathing rate; and
6) playing the first and second audio signals to the patient for a period of a treatment session, wherein the first and second audio signals are played simultaneously for at least a portion of the treatment session, and wherein the second audio frequency is slower than the patient breathing rate or is slowed during the treatment session to a rate that is slower than the patient breathing rate.

2. (canceled)

3. The system of claim 1, wherein the first audio signal is a human breathing sound.

4. The system of claim 1, further comprising playing simultaneously with the first and second audio signals, during at least a portion of the treatment session, a third audio signal comprising a binaural beat created from two tones, wherein the two tones have frequencies separated by a gap that is a transposition of the exceptional sound frequency, in the range of 0.1 to 30 Hz, and wherein a mean of the two tones is the exceptional sound frequency.

5. The system of claim 1, wherein the first and second audio signals are played at a volume dependent on the patient stress level, and wherein the volume is increased during the treatment session as the patient stress level drops.

6. The system of claim 1, wherein playing the first and second audio signals comprises playing one audio signal by itself for a first period of the treatment session, then playing two audio signals simultaneously for a second period of the treatment session, and then playing the one audio signal by itself for a third period of the treatment session.

7. The system of claim 1, further comprising playing simultaneously with the first and second audio signals, during at least a portion of the treatment session, a third audio signal comprising binaural 3D nature sounds.

8. The system of claim 1, further comprising playing simultaneously with the first and second audio signals, during at least a portion of the treatment session, a third audio signal comprising the exceptional energy sound frequency.

9. The system of claim 1, further comprising playing, simultaneously with the first and second audio signals, during at least a portion of the treatment session, a third audio signal comprising the exceptional sound frequency, wherein the third audio signal is spatially varying with an oscillation corresponding to a rate that is similar or lower that a frequency of a monitored heart rate variability parameter, an EEG signal parameter, or the patient breathing rate.

10. The system of claim 1, wherein the exceptional energy level frequency is identified by frequency analysis of the patient's speech.

11. The system of claim 1, further comprising characterizing a responsiveness of the patient's vagus nerve to the audio signals and responsively adjusting a frequency and/or volume of the audio signals.

12. The system of claim 1, further comprising delivering to the patient tactile and/or visual stimulation during the treatment session.

13. The system of claim 1, further comprising adjusting a volume of the audio signals to the patient's schedule and environment.

14. The system of claim 1, further comprising analyzing accumulated data from multiple patients to enhance the derivation of the audio signals.

15. The system of claim 1, further comprising providing a user interface for presenting bio-feedback, wherein the user interface includes visual, gaming and/or social network features.

16. The system of claim 1, wherein the measured physiological characteristics further comprise EEG signals.

17. The system of claim 1, further comprising scanning a range of frequencies and measuring at each frequency one or more physiological characteristics of the patient indicative of stress reduction to determine an optimal frequency of a third audio signal to play during the treatment session.

18. The system of claim 1, further comprising implementing eye movement desensitization and reprocessing (EMDR) procedures of eye movement monitoring during the treatment session.

19. A method for patient treatment, implemented by a processor having associated non-transient memory including instructions that when executed cause the processor to perform the method, wherein the method comprises:
1) receiving sounds vocalized by a patient;
2) determining, from the vocalized sounds, an exceptional frequency that is either a prominent or attenuated frequency;
3) deriving a first audio signal including the exceptional frequency;
4) measuring one or more physiological characteristics indicative of a patient breathing rate and of a patient stress level;
5) deriving a second audio signal from the patent breathing rate, wherein the second audio signal is a repeated and/or spatially oscillating at a second audio frequency no greater than the patient breathing rate; and
6) playing the first and second audio signals to the patient for a period of a treatment session, wherein the first and second audio signals are played simultaneously for at least a portion of the treatment session.
